(19) United States
(12) Patent Application Publication
(10) Pub. No.: US 2010/0196495 A1
(43) Pub. Date: Aug. 5, 2010

(54) DELIVERY OF FLU ANTIBODIES TO SURFACES IN CONTACT WITH AIR

(75) Inventors: Grant Thomas RAWLIN, Victoria (AU); Gottfried LICHTI, Victoria (AU); Roy Michael ROBINS-BROWNE, Victoria (AU)

Correspondence Address: PARFOMAK, ANDREW N.;NORRIS MCLAUGHLIN & MARCUS PA, 875 THIRD AVE, 8TH FLOOR, NEW YORK, NY, 10022, US

(73) Assignee: IMMURON LIMITED, Victoria (AU)

(21) Appl. No.: 12/576,467

(22) Filed: Oct. 9, 2009

Related U.S. Application Data
(63) Continuation-in-part of application No. PCT/AU2008/000509, filed on Apr. 11, 2008.
(60) Provisional application No. 60/907,621, filed on Apr. 11, 2007.

(51) Int. Cl. A61K 39/44; A61K 39/42; A61K 9/14; A61P 31/16

(52) U.S. Cl. 424/491; 424/159.1; 424/178.1

(57) ABSTRACT

The invention relates to a method, composition and inhaler system for treatment or prophylaxis of influenza infection in one or more subjects by applying to a surface selected from air filters, sick room surfaces and respiratory mucosal membranes at least one immune material selected from antibodies and fragments thereof which bind a least one Influenza A antigen selected from the group consisting of H1, H3 and H5, the immune material being derived from hyperimmune milk products such as hyperimmune colostrum said hyperimmune milk products being prepared by inoculation of mammals with antigen composed of a least one Influenza A antigen selected from H1, H3 and H5.

### PRIORITY CLAIM

This application is a continuation-in-part of International Patent Application No. PCT/AU2008/000509, filed on Apr. 11, 2008, which, in turn, claims priority from U.S. Provisional Patent Application No. 60/907,621, filed on Apr. 11, 2007. The contents of the international application and the US provisional patent application are herein incorporated by reference.

### FIELD OF THE INVENTION

This invention relates to a method of treating or inhibiting influenza, to a composition for use in such treatment, to the use of such composition in manufacture of a medicament for treatment or inhibition of influenza and to materials and surfaces modified with the composition.

### BACKGROUND

Epidemic influenza is extremely prevalent on a worldwide scale and is caused by a group of viruses known as Influenza A. Epidemic influenza virus is transmitted primarily in aerosols. These aerosols may be generated by coughing or sneezing. The aerosol particles retain the viable virus and deposit the virus on the mucosal surfaces thereby initiating infection. Infection is thus initiated in the entire respiratory tract. Surfaces that have a particular propensity for uptake of virus-laden aerosol are surfaces in proximity to regions of turbulent airflows. In a human lung the mucosal surfaces in proximity to regions of turbulent inflow are not alveolar surfaces or terminal or quasi-terminal bronchioles (where the air flow is laminar), but mucosal surfaces 3 to 4 branch points away from the trachea.

Antiviral agents for treatment of Influenza A virus include chemical moieties such as amantadine, oseltamivir, zanamavir or rimantadine. However, resistance against these agents is beginning to develop, there is a relatively narrow therapeutic window and they are very expensive and difficult to manufacture at large scale. Therefore they are of limited effectiveness for use in a wide population. Most of these agents are transported via the blood stream so the local concentration at the infection site is relatively low.

The use of parenteral injection of antisera (passive immunity) against viral and bacterial infection has been known for many years. Records of the use of injectable antibodies to successfully treat ‘Spanish’ flu have been found dating from the 1920s: Luke et al, 2006 in Annals of Internal Medicine, Volume 145 Number 8 pp 599-609. This describes the use of human convalescent blood and plasma to treat Spanish influenza pneumonia.

Of the influenza viruses, the Influenza A virus undergoes a significant change in morphology from time to time, and is important in terms of the damage it causes to human health.

Treatment with an inactivated vaccine has been attempted—this is the basis for commercial flu vaccines e.g. vaccines sold by CSL in Australia. However, this vaccine does not have a sufficient effect to sustainably produce antibodies, and thus cannot completely prevent the spread of infection in all patients. Killed vaccines often do not give good protection to old and young people and people with compromised immune systems.

Injectable treatments of antibodies (usually intravenous) have proved unsuitable for population prophylaxis as they generally require close medical supervision due to the high risk of anaphylaxis. Furthermore use protocols for intravenous antibody injection generally require ready access to adrenalin and dexamethasone to treat acute anaphylaxis if it occurs.

Hilty et al, (1999 U.S. Pat. No. 5,922,344) describes a method in which polyclonal human plasma flu antibodies are used orally. Oral treatments in the case of most influenza viruses are not effective as they do not reach the area where the virus is present.

Ramisse et al, (1998, Clinical Experimental Immunology 111:583-587) details experiments for examining prophylaxis against influenza in which plasma polyclonal anti-influenza antibodies are delivered to the respiratory tract of mice.

Mozdzanowska et al, (2003 in Journal of Virology Vol 77, No 15 pp 8322-8328) describes experiments aimed to resolve influenza virus infection in mice in which a Fab fragment of monoclonal antibodies is used.

Palladino et al, (1995 Vol 69, No 4 pp 2075-2081) describe experiments in which monoclonal antibodies were used to cure influenza disease in mice.

Recently parenteral injection of human antibodies has been used for prophylaxis against a number of respiratory diseases such as respiratory syncytial virus. Such passive protection has been found useful in prophylaxis but is generally considered less effective when administered therapeutically.

Human IgG has been used in studies examining the efficacy of passive parenteral administration with some success in prophylaxis in animal models.

There is a need for an effective method of neutralizing infectious flu particles which results in inhibition or treatment of influenza and is in a convenient and safe form for use in treatment and inhibition of influenza infection.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY

We have found that certain compositions comprising anti-flu antibodies and fragments thereof derived from milk products such as colostrum can be applied to surfaces in contact with air to provide effective treatment or inhibition of influenza.

Accordingly, we provide a method for treatment or inhibition of influenza infection in one or more subjects comprising applying to a surface (preferably selected from air filters, sick room surfaces and respiratory mucosal membranes) at least one immune material selected from antibodies and fragments thereof which bind a least one Influenza A antigen selected from the group consisting of H1, H3 and H5 said immune material being derived from hyperimmune milk products (such as hyperimmune colostrum) said hyperimmune milk products being prepared by inoculation of mammals with antigen comprising at least one Influenza A antigen selected from the group consisting of H1, H3 and H5.

The air filters may be personal air filters such as may be worn over the nose and mouth of the subjects or the filters may be building or air conditioner filters in buildings inhabited by the subjects.

In a particularly preferred aspect the invention provides a method of treatment or inhibition of influenza infection in a human subject comprising administering by inhalation a composition comprising immune material selected from influenza antibodies and fragments thereof which bind a least one Influenza A antigen selected from the group consisting of H1, H3 and H5 said immune material being derived from hyperimmune milk products (such as hyperimmune colostrum) prepared by inoculation of mammals with antigen comprising a least one Influenza A antigen selected from the group consisting of H1, H3 and H5.

The inhaled immune material is capable of binding to at least one epitope from the H1, H3 or H5 antigens of influenza A.

In a further aspect we provide a composition for the treatment or inhibition of influenza infection by inhalation comprising at least one immune material selected from flu antibodies derived from mammalian (preferably bovine) milk or colostrum and fragments thereof which bind at least one Influenza A antigen selected from the group consisting of H1, H3 and H5.

We have found that the immune material prepared by inoculation of mammals with Influenza A antigen comprising at least one of H1, H3 and H5 is significantly enhanced for use in the treatment or inhibition of Influenza infection if the antigen further comprises lipopolysaccharide (LPS). Thus, in a further aspect we provide a composition for the treatment or inhibition of influenza infection by inhalation comprising at least one immune material selected from flu antibodies derived from mammalian (preferably bovine) milk or colostrum and fragments thereof which bind at least one Influenza A antigen selected from the group consisting of H1, H3 and H5.

In a particularly preferred embodiment the immune material is absorbed onto a solid carrier for administration by (dry powder) inhalation.

In a particularly preferred embodiment the composition is in the form of a unit dose for inhalation comprising at least 1 mg of immune material, preferably at least 5 mg and more preferably 10 mg of immune material.

### BRIEF DESCRIPTION OF THE FIGURES

FIG. 1. High-titred antibodies from bovine colostrum prevent infection (100% protection) with virulent influenza for 24 hours following a single dose. Viral titres are given in log₁₀ pfu/ml.

FIG. 2. High-titred antibodies from bovine colostrum prevent infection (100% protection) with virulent influenza for 5 days following a single dose. Viral titres are given in log₁₀ pfu/ml.

FIG. 3. High-titred antibodies from bovine colostrum prevent infection (100% protection) with virulent influenza for 48 hours following a single dose. Viral titres are given in log₁₀ pfu/ml.

FIG. 4. High-titred antibodies from bovine colostrum prevent infection (100% protection) with virulent influenza for 72 hours following a single dose. Viral titres are given in log₁₀ pfu/ml.

FIG. 5. Reduction of viral loads following treatment with specific immune IgG according to the present invention. A significant effect was observed at 5 days post infection; 4 days after a single IgG treatment. Viral titres are given in log₁₀ pfu/ml.

FIG. 6. Reduction of pulmonary viral loads following treatment with specific immune IgG according to the present invention. A significant effect was observed at 5 days post infection; 4 days after a single IgG treatment. Viral titres are given in log₁₀ pfu/ml.

FIG. 7. Protection from death and prevention of weight loss following treatment with specific immune IgG according to the present invention.

### DETAILED DESCRIPTION

The term colostrum where used herein includes colostral milk; processed colostral milk such as colostral milk processed to partly or completely remove one or more of fat, cellular debris, lactose and casein; and colostral milk or processed colostral milk which has been dried by for example, freeze drying, spray drying or other methods of drying known in the art. Colostral milk may be taken from a mammal such as a cow within five days after parturition.

Preferably the mammalian colostrum has been processed using a detailing operation, more preferably using a defatting operation and an operation to remove cellular debris, still more preferably a defatting operation, an operation to remove cellular debris and an operation to remove salts, sugars, other low molecular weight entities and some water.

Lipopolysaccharide (LPS) are used in one embodiment as an antigen co-administered with an Influenza A antigen. Co-administration may involve administration of a mixed antigen or separate administration of LPS and Influenza A antigen. LPS is a major component of the outer membrane of Gram-negative bacteria, contributing greatly to the structural integrity of the bacteria, and protecting the membrane from certain kinds of chemical attack.

The hyperimmune milk product may be in dried form. Further, components, such as protective agents, when used may be intimately mixed before, during or after the drying process.

Throughout the description and the claims of this specification the word “comprise” and variations of the word, such as “comprising” and “comprises” is not intended to exclude other additives, components, integers or steps.

The effectiveness of such a composition of immune material for both treatment and inhibition of influenza was not expected as existing vaccines and treatments have involved parenteral administration which has generally been accepted as the effective method of treating or preventing respiratory disease.

Without wishing to be bound by theory, the present inventors believe the effectiveness of treatment and inhibition by inhalation may result from the mechanism for transmission of Influenza A virus between adjacent cells. It is believed by the present inventors that Influenza A virus is transmitted from one cell to another via the mucous layer covering the respiratory mucosa—rather than via the blood in a viraemic phase. The respiratory mucosa represents a high surface area surface due to the presences of structures such as nostril hairs, turbinate passages, sinuses, ciliated tubular upper airways (trachea), branched ciliated secondary airway (primary bronchi), multiple branching airways (secondary bronchi) and other high surface area surfaces. This proposed mechanism for transmission of influenza A from one cell to another is not shared by most viruses that infect the respiratory tract and cause disease, such as respiratory syncytial disease, adenovirus, rhinovirus, pandemic flu and calicivirus.

One embodiment of the invention provides a method of treatment or prophylaxis of influenza particularly influenza A. Milk products, particularly colostrum provide the source of immune material such as antibodies used in the present invention for providing passive immunity or treatment of influenza. It has long been recognised that immunization of ungulate animals with antigens during pregnancy can lead to the production of high levels of antibodies in colostrum.

This is reported as useful in providing passive immunization of calves against bacterial infection in British Patent 1211876 and Singh uses a similar approach in U.S. Pat. No. 3,911,108 to produce pig feed containing antibodies to protect baby pigs against transmissible gastroenteritis. U.S. Pat. No. 3,911,108 further reports the use of immunological milk from cows immunized with a bacterin vaccine which is said to provide immunity to people drinking same.

Many respiratory diseases such as those caused by Respiratory Syncitial Virus, Yersinial Pestis, Bacillus anthracis or cold virus, are transferred via the blood once infection has commenced. Inhalation wouldn't be expected to be effective in treating viral infections.

The present invention utilizes immune material obtained by vaccinating mammals particularly ungulate animals with influenza virus or parts thereof and collecting milk products, particularly colostrum from the vaccinated mammals. The milk products enriched in antibodies to influenza are used to neutralise influenza virus on surfaces and in particular surfaces of air filters, of a sick room and of the mucous membranes of the respiratory tract.

As used herein, the terms “antibody”, “antibodies” and the like include any monospecific or bispecific molecule comprising a portion of the light chain variable region and/or the heavy chain variable region to effect binding to the epitope to which the antibody has binding specificity. It will be understood that as the immune material is raised by vaccination of mammals it will contain polyclonal antibodies. Antibodies, as used herein, may also include polyclonal, humanized, anti-idiotypic, chimeric or single chain antibodies. Exemplary antibodies and fragments thereof that may be prepared according to this aspect of the invention include intact immunoglobulin molecules, substantially intact immunoglobulin molecules and fragments that contain a paratope.

Fragments, as used herein, typically include a portion of an antibody molecule that retains the ability to specifically bind to an antigen (e.g., an influenza antigen) and include, but are not limited to, Fab, Fab′, F(ab′)₂ and F(v). Antibody fragments may be obtained from antibodies such as described above by methods such as digestion by enzymes, such as pepsin or papain and/or by cleavage of the disulfide bridges by chemical reduction. Single chain antibodies are also intended to be encompassed within the term “fragment”. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Still further, an antibody or fragment thereof may be part of a larger immunoadhesion molecules, formed by covalent or non-covalent association of the antibody or fragment thereof with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

One embodiment of the invention provides a composition and method of treatment or inhibition of Influenza A involving use of a polyclonal antibody material or a fragment thereof, derived from a milk, particularly colostrum. The antibody material or fragment thereof is derived by inoculation of mammals, particularly bovine mammals, with an antigen comprising at least one of the H1, H3 or H5 antigens of influenza A. The composition may be in the form of a dispersion or solution in a suitable liquid carrier and delivered in spray form such as by a metered dose inhaler (MDI) to the respiratory tract. Alternatively and preferably the composition is delivered to the respiratory tract in particulate form by being dispersed in air using, for example, a dry powder inhaler (DPI).

Alternatively the antibody material may be adsorbed onto a porous substrate such as a filter, face mask, tissue paper

It is preferred that the immune material comprising anti-flu antibodies or fragments thereof which bind a least one Influenza A antigen selected from the group consisting of H1, H3 and H5 further comprises components derived from mammalian milk particularly colostrum. Examples of such components include for example one or more components selected from the group consisting of casein, lactose and growth factors including insulin-like growth factor or transmissible growth factor. In one embodiment, the compositions comprising anti-flu antibodies or fragments thereof further comprise agents designed to preserve the bioactivity of the antibodies or fragments thereof. Such bioshielding agents include agents disclosed in our International Patent Publications WO 2003/080082 and WO 2004/078209, the contents of which are incorporated by reference.

Colostrum may, if desired, be added to the antibodies or fragments thereof which bind a least one Influenza A antigen selected from the group consisting of H1, H3 and H5. In this embodiment the antibodies or fragments thereof may be purified to at least partly separate them from other milk products and subsequently colostrum added to provide protection of the biological activity of the immune material from the hostile environment of the respiratory tract. The colostrum used in providing the protective function may be defatted and purified.

The components may be intimately mixed before, during or after the drying process.

The antibodies or fragments thereof which bind a least one Influenza A antigen selected from the group consisting of H1, H3 and H5 comprise IgG. Preferably the total of IgA, IgE and IgM moieties are less than 20% of total Immunoglobulin and the IgG component constitutes at least 50% of said moieties and more preferably at least 70% of said moieties. The antibodies of the present invention may be of any of the different subclasses or isotypes of immunoglobulin, including, but not limited to, IgA, IgG, IgE IgM (or any of the other subclasses) or any combination thereof.

In one preference, the antibody material is effective in binding at least two Influenza A antigen selected from the group consisting of H1, H3 and H5 from the H1, H3 or H5 antigens of influenza A, and more preferably all three.

In one particularly preferred aspect the invention provides a treatment of influenza A infection in a human subject comprising administering by inhalation a composition comprising immune material selected from antibodies and fragments thereof which bind a least one Influenza A antigen selected from the group consisting of H1, H3 and H5 said immune material being derived from hyperimmune milk products (such as hyperimmune colostrum) prepared by inoculation of mammals with antigen comprising a least one Influenza A antigen selected from the group consisting of H1, H3 and H5. In this embodiment the composition is administered so as to come in contact with an airway surface of a human subject in the upper respiratory tract, preferably an airway surface within 3 or 4 branch points of the trachea.

In another embodiment, the surface is any surface in a room frequented by subjects with influenza A (hereinafter referred to as a sick room).

In one preference, the surface is a filter on which the immune material is adsorbed. The filter may be used to inhibit infection of people with the influenza A virus which may be present in the airstream. Preferably the airstream is made to develop as a turbulent stream in proximity to the filter.

In one embodiment, the antibody titre of the composition as herein described that neutralises at least one of H1, H3 and H5 antigens of influenza A virus is greater than 1:16, preferably greater than 1:80.

In an embodiment of the invention the polyclonal antibody is contained in colostrum which has been defatted, cleansed of cellular debris and filtered to remove material of molecular weight less than 5 kD.

Where it is used in the composition, immune material fragment which bind a least one Influenza A antigen selected from the group consisting of H1, H3 and H5 may be an F(Ab) or F(Ab)′2 fragment.

The antibody preparation raised in milk may be affinity purified against influenza A antigens, preferably H antigens, more preferably against at least one of H1, H3 or H5 antigens.

In one preference, the polyclonal antibody is collected and retained as a separate specimen from each individual cow until after quality control is performed.

In one preference, the colostrum comprising the polyclonal antibody is first, second or third milking colostrum, preferably first or second milking, more preferably first.

In one preference, the antibodies or fragments thereof are provided in finely divided form, preferably in association with a carrier particle or other respiratory adjuvant. Preferably the adjuvant comprises lactose, albumin or other inhalation excipients regarded as GRAS (Generally Regarded as Safe) by the Food and Drug Administration of the USA.

In one preference, the antibodies or fragments thereof are made by vaccinating a cow with killed whole influenza virus using an adjuvant several times during the 3 months before calving. Preferably the adjuvant is chosen from the set of Alum, an oil-in water-in oil adjuvant or a water in oil in water adjuvant. Preferably the adjuvant comprises one of the Montenide product family sold by Seppic of France. The colostrum is harvested after the calf is born, and is preferably stored frozen until processing. Preferably the liquid colostrum is skimmed of fat, pasteurised and ultrafiltered in accordance with protocols described in PCT/AU 2004/000277 which is incorporated by reference. Drying may involve freeze drying or spray drying, preferably freeze drying. Preferably the immunogen comprises at least one haemagglutinin epitope, more preferably at least two haemagglutinin epitopes, preferably the epitopes are chosen from the set H1, H3, H5.

In one preference, antibody fragments are chosen from the group consisting of F(Ab) fragments, F(Ab)′2 fragments, fragments generated by enzymatic cleavage of immunoglobulin moieties by pepsin, trypsin, chemotrypsin, and papain.

In one embodiment, the antibodies or fragments are derived from bovine mammalian colostrum, and colostrum samples collected from individual animals are kept separate from each other until quality control tests have been conducted on the individual samples.

The immune material may be combined with one or more auxiliaries or auxiliaries to aid formulation or enhance the stability of the composition in the environment in which it is to be used.

We have found that the immune material prepared by inoculation of mammals with Influenza A antigen comprising at least one of H1, H3 and H5 is significantly enhanced for use in treatment or inhibition of Influenza if the antigen further comprises lipopolysaccharide (LPS). The lipopolysaccharide may be in the form of killed gram negative bacteria, attenuated gram negative bacteria, or LPS separated from cell walls of gram negative bacteria. LPS may be separated, at least in part, by a range of methods using for example heat, detergents, lysis or mechanical means. Methods of separating LPS from cell walls of bacteria are described in our application WO/2004/078209 (with reference to separation of O-antigen) the contents of which are herein incorporated by reference. In particular the preferred method of separating LPS from cell walls is by application of shear. The LPS antigen used in vaccination can be separated from the bacterial cell walls by application of an effective amount of shear, homogenisation or heat or by effective combinations thereof.

In one embodiment of the invention, which we have found to be particularly efficacious, the antigen used to inoculate the mammal to enable a high titre of polyclonal antibodies is in the form of a split antigen. The split antigen in accordance with this embodiment may be prepared for example from killed H1N1 influenza virus treated with detergents to provide a split-virus vaccine. When a virus is treated with detergent, a sub-unit or purified surface antigen vaccine is provided which is enriched in HA and contains only residual internal structural protein. A general procedure for preparation of split antigen is described in Immunisation Safety Review, Dathleen R Stratton 2004, National Academies press, p37.

In one embodiment, the antibodies or fragments thereof are provided in the form of an inhalational dose, and at least 1 mg of antibody or antibody fragment is used in each inhalational dose. Preferably at least 3 mg, more preferably at least 10 mg.

The antibodies or fragments thereof may be provided in milled particulate form of the immune material wherein the average particle size is less than 20 microns, preferably less than 10 microns, more preferably less than 5 microns. Once small particles have been produced, the micronized substance may be blended with an excipient. Examples of suitable carriers may include one or more carbohydrate, such as fructose, glucose, galactose, sucrose, lactose, trehalose, raffinose, melezitose; alditols, such as mannitol and xylitol; maltodextrins, dextrans, cyclodextrins, amino acids, such as glycine, arginine, lysine, aspartic acid, glutamic acid and polypeptides, such as human serum albumin and gelatin. To mask the unpleasant taste of some inhaled drug compounds, flavoring particles containing maltodextrin and peppermint oil may be incorporated into dry powder formulations. Large sized particles increase mouth deposition and reduce lung deposition. Lactose is a particularly preferred carrier.

The carrier particles are typically relatively large such as on the order of 50 to 120 microns, or approximately 50 times bigger than the milled particles containing immune material. These carrier particles help to facilitate the dispersion of the small particles and allow precise filling into the dry powder inhaler (DPI) powder storage system in a reproducible manner. Milled antibody is blended with lactose at concentrations ranging from <1 to 50% by weight. Finally, the blend is filled into the powder storage systems of an inhaler at weights ranging from approximately 3-25 mg. Preferred powder storage systems are shown in U.S. Pat. Nos. 5,492,112; 5,645,051; 5,622,166; and 5,921,237, incorporated herein by reference. The inhaler and storage systems shown in U.S. Pat. Nos. 5,921,237 and 5,622,166, incorporated herein by reference, are appropriate for use with vaccines. In these systems, a dry powder formulation is sealed into a foil blister that protects the powder from exposure to high humidity, reduces the risk of contamination, and can prevent inactivation of the vaccine by sunlight. The process of preparing dry powder blends for aerosol delivery involves three basic steps.

Size reduction may be accomplished by a variety of techniques including spray drying, precipitation from supercritical fluids, and jet milling or micronization. Preferably, jet milling is used. This technique uses high pressure, high velocity gas to cause particle to particle attrition to generate small particles at high efficiencies. Multidose DPIs may be used with disposable cassettes or foil blister disks, strips or unit dose blisters to deliver many doses, contributing to the cost effectiveness of this approach as compared with syringes, particularly single use syringes. These DPI's may be provided with disposable mouthpieces that can be used in mass dosing campaigns. Alternatively, unit dose DPIs with vaccine sealed in the aerosolization chamber can be used.

The inhaler may have a body, a mouthpiece and an airflow passage. A restrictor plate may be used having flow control openings in the airflow passage opposite from the mouthpiece. A dose of dry powder vaccine sealed by a foil strip or capsule is received within the body of the inhaler. In use, the foil strip or capsule is pulled out or back, peeling or breaking open blister formed around the dose or a capsule is received in a chamber provided with means such as a sharp object for piercing the capsule to release the powder which may occur in response to actuation by the user to urge the sharp object into the capsule or blister by means of a trigger lever, button or the like. The subject inhales on the mouthpiece, and the dose is drawn into the lungs.

Alternatively, or in addition, the inhaler may be provided with means for actively generation an air flow in response to actuation by manual operation of the user or commencement of the inhalation process by the user.

In one preference the antibodies or fragments are treated to diminish issues of allergic response. Treatment may involve removal of allergenic moieties or the inclusion of adjuvants designed to diminish allergenicity.

In one preference the antibodies or fragments are treated by affinity purification methods to remove antibodies or fragments that bind to non-target material.

In one preference, the antibodies or fragments are deposited on the surface of a face mask, or within the fabric of a face mask.

In one preference, the antibodies or fragments are dissolved or dispersed in a liquid which is then used in a nebulising device or to provide an antibody aerosol which is released into the atmosphere of a hospital ward or sick room or other polluted atmosphere containing flu virus.

In one preferment, the antibody aerosol is directed into an air stream comprising polluted air which is then passed over or through a surface, for example a porous surface or a hairy surface or some other surface having an extended surface area. In this case the role of the surface is to capture both flu viruses or virions and also antibody moieties, leading to at least partial neutralisation of the polluted air stream.

Some of the advantages of the invention are:
The antibodies or fragments thereof are applied to surfaces in contact with air are less likely to allow disease transmission than are products derived from blood,
Dairy-derived antibodies or fragments thereof are more likely to be non-irritant than blood product derived antibodies. Milk aerosols are commonly encountered by humans with no ill effects (for example, sucking the last bit of a milk shake through a straw creates a milk aerosol).
Dairy derived antibodies have a better animal ethics profile than blood derived antibodies.
Bovine colostrum antibodies are more homogeneous than bovine plasma antibodies in that the former comprise mainly IgG whilst the latter comprise elevated levels of IgA, IgE and IgM.
In a production environment, more antibodies can be harvested from one cow in a single operation than from blood from a living animal.
When harvesting colostrum it is feasible to conduct individual animal quality control using methodology consistent with efficient dairy practice.
Dairy derived polyclonal antibodies bind their target through multiple binding sites so a greater spectrum of cross reactivity can be achieved compared to monoclonal antibodies.
Production protocols for dairy derived polyclonals are much simpler than for monoclonals, leading to reduced infrastructure and production costs.

The invention will now be described with reference to the following examples. It is to be understood that the examples are provided by way of illustration of the invention and that they are in no way limiting to the scope of the invention.

### Example 1

A composition of the invention may be prepared in accordance with the following description. 15 micrograms of killed H1N1 influenza virus (PR8 mouse adapted strain) is mixed with 1 ml of Montenide ISA adjuvant and injected 3 times into a pregnant dairy cow at 2 week intervals, with the last injection occurring 1 month before calving.

Colostrum is harvested. Raw colostrum tested as 10^{2.7} titre using a cell-based virus neutralisation test against H1N1. Antibody is purified from the protein fraction of the colostrum using a Protein A column.

### Example 2

Adult male mice (Balb-c) are anaesthetised, treated then challenged with a lethal dose of H1N1(PR8) influenza by the nasal route. 10 mice are treated with 25 micrograms of purified antibody delivered by a liquid drop to the nose before challenge with the virus. 5 virus control animals are treated with Phosphate buffered saline before virus challenge.

90% of the mice treated with antibody preparation are found to survive for 14 days, while all of the virus control group show severe symptoms leading to 100% mortality.

### Example 3

Flat filter mask material is immersed in a 0.1% solution of purified polyclonal antibodies (procured as described in Example 1) and is lyophilised in a freeze drier, and is subsequently fabricated into a filter mask.

### Example 4

Colostrum (procured as in Example 1) is defatted and purified of cellular debris, followed by lyophilization. The freeze dried powder is milled and a fine fraction (sub 5 micron) collected and mixed/tumbled well with carrier particles of lactose (60 micron in size). The weight ratio of fine colostrum to lactose is 1:10. The mixed powder is suitable for dispensing in a powder inhaler.

### Example 5

A composition of the invention may be prepared in accordance with the following description. 15 micrograms of killed H1N1 influenza virus (PR8 mouse adapted strain), is treated with detergents to provide a split-virus vaccine in accordance with procedures previously reported for preparation of split viral vaccines. When a virus is treated with detergent, a sub-unit or purified surface antigen vaccine is provided which is enriched in HA and contains only residual internal structural protein (Immunisation safety review, Dathleen R Stratton 2004, National Academies press, p37). The resultant split virus material is mixed with 1 ml of Montenide ISA adjuvant and injected 3 times into a pregnant dairy cow at 2 week intervals, with the last injection occurring 1 month before calving.

Colostrum is harvested. Antibody is purified from the protein fraction of the colostrum using a Protein A column. At 5 mg/kg, anti-flu IgG showed 100% neutralisation at 50 pfu and 92.9% neutralisation at 500 pfu of PR8 virus in a neutralisation test based on plaquing MDCK monolayers.

### Example 6

10 Adult male mice (Balb-c) are anaesthetised, challenged with a non-lethal 50 pfu dose of H1N1(PR8) influenza by a drop put on the nares. 5 mice are treated with 150 micrograms of purified antibody derived from samples described in Example 1 at 8 hours after infection. The treatment was delivered by a liquid drop to the nares. 5 virus challenged control animals were treated with Phosphate buffered saline 8 hours after virus challenge. The body weights of the mice were measured and averaged daily over 5 days. Challenge control mice lost 15% of body weight compared to treated mice which lost 2% at the end of the 5 days.

### Example 7

10 Adult male mice (Balb-c) are anaesthetised, challenged with a lethal 500 pfu dose of H1N1(PR8) influenza by a drop put on the nares. 5 mice are treated with 50 micrograms of purified antibody derived from samples described in Example 1 at 2 hours after infection. The treatment was delivered by a liquid drop to the nares. 5 virus challenged control animals were treated with Phosphate buffered saline 2 hours after virus challenge. Mouse mortality was measured over a period of 9 days. After 9 days, all the control mice were dead but 60% of treated mice survived.

### Example 8

Adult male mice (Balb-c) are challenged with a non-lethal 50 pfu dose of H1N1(PR8) influenza by a drop put on the nares. 5 mice are treated with 50 micrograms of purified antibody derived from samples described in Example 4 daily for 4 days by placing a drop on the nares. 5 virus challenged control animals were treated with Phosphate buffered saline in a similar manner. On Day 5 the mice were killed and their noses were collected for virus isolation.

The virus titre of challenge control mice was significantly higher (p=0.0317) than those of the treatment group (av 1.2 log₁₁₀ pfu/ml v 3.0 log₁₀ pfu/ml).

### Example 9

This example describes a process by which LPS antigen may be prepared and a process for forming hyperimmune material by co-inoculation of the LPS antigen and Influenza A antigen in separate intramuscular injections:

### Part A

An LPS adjuvant for use in inoculation of bovine animals to enhance the activity of antibodies and fragments thereof in colostral milk resulting from co-inoculation with Influenza Antigen may be prepared by the procedure of Example 1 of WO 2004/078209 wherein the wall antigen is E. Coli 078 and the pilus antigen is CFA1. The procedure used may be as follows:

Day 0 (Step A) Strain Rejuvenation. The strain to be rejuvenated is E. coli H10407 (Taurchek et al, PNAS USA 2003, 99: 7066-7071). Take 2 CFA plates (Evans et al, Infect Immun 1977; 18: 330-337) from the media refrigerator and place them in the biological safety cabinet. Remove the vial containing E. coli H10407 from the liquid nitrogen tank and place it in the biological safety cabinet. Open the vial and use a sterile loop to remove a small quantity of frozen material. Streak this material onto CFA plates. Place the “rejuvenation plates” in the 37° C. incubator overnight under aerobic conditions.

Day 1 (Step B) Inoculation of “starter suspension”. Examine each “rejuvenation plate” for pure growth. If pure growth is present proceed. Working in the biological safety cabinet, remove several colonies from one “rejuvenation plate” with a sterile loop and inoculate a McCartney bottle containing 20 mL of phosphate buffered saline (PBS) pH 7.2. Use McCartney bottles and PBS that have been sterilised by autoclaving.

(Step C) Inoculation of “vaccine plates”. Inoculate 50 pL of “starter suspension” onto each of multiple CFA plates (microbiological nutrient plates formulated to enable production of CFA).

CFA plates are prepared using 1% casamino acids (BD Difco) and 0.15% yeast extract (Oxoid) in 2% agar containing 0.005% MgS04 (anhydrous) and 0.0005% MnCl2 (tetrahydrate), as described in media preparation (Evans et al., Infect Immun 1977; 18: 330-337). Place the “vaccine plates” in the 37° C. incubator for 18-24 hours under aerobic conditions.

Day 2 (Step D) haemagglutination test on “vaccine plates” to test for pilus production. Carry out the Haemagglutination test (Evans et al., Infect Immun 1977; 18: 330-337). Test one “vaccine plate” for each strain to be used in the batch. If positive proceed.

(Step E) Washing of “vaccine plates”. Remove the “vaccine plates” from the incubator. Check each one for contamination and reject any affected plates.

Working in the biological safety cabinet, use 1.5-2.0 mL of sterile 0.1M sodium phosphate buffer (pH 7.2) to wash the bacterial growth from the surfaces of the “vaccine plates” into a sterile Schott bottle. Pre-cool the buffer on ice before use. Add sodium azide to a final concentration of 0.05% to the “vaccine washings”. Keep the “vaccine washings” on ice for at least 30 minutes before commencing homogenisation.

(Step F) Enumeration of “Vaccine Washing”. Carry out enumeration of the “vaccine washings”. Ensure that the material has been thoroughly agitated to disperse all clumps and that the dilutions chosen are appropriate to the degree of concentration of bacterial cells in the washings for the batch being manufactured.

(Step G) Purity sampling. Assemble sufficient materials for purity testing each “vaccine washing” (i.e. 3 HBA Plates, 3 TSA Plates and 3 MAC plates).

Working in the biological safety cabinet, streak out each “vaccine washing” onto 3 HBA, 3 TSA and 3 MAC plates, plating for single colonies. Place the plates in the 37° C. incubator under aerobic conditions.

Day 3 (Step H) First reading of the “purity test plates”. Carry out the first reading on the “purity test plates”. If the plates contain only colonies of E. coli proceed.

(Step I) Homogenisation of “vaccine washing”. Homogenise the “vaccine washing” in the homogeniser for a total of 15 minutes at one minute intervals, with one minute of cooling in an ice-bath between each interval. Centrifuge the “homogenised vaccine washing” in the high speed centrifuge at 12,000×g for 20 minutes at 4° C. Keep the supernatants (HVW super 1) and store at 4° C. for 1-3 days.

Day 4 (Step J) Second reading of the “purity test plates”. Carry out the second check on the purity test plates. If the plates contain only pure colonies of E. coli proceed.

Day 6 (Step K) Separation of LPS fraction Centrifuge the “HVW super 1” in the high speed centrifuge at 12,000×g for 20 minutes at 4° C. Keep the supernatant (HVW super 2).

Add sterile saturated ammonium sulphate to the “HVW super 2” slowly over 1 hour until 20% saturation is reached. Stir the “HVW super 2” on a magnetic stirrer while adding the saturated ammonium sulphate. At the end of the hour allow the material to equilibrate for 30 minutes.

Centrifuge the “HVW super 2” in the high speed centrifuge at 12,000×g for 20 minutes. Keep the supernatant (HVW super 3).

Add sterile saturated ammonium sulphate to the “HVW super 3” slowly over 1 hour until 40% saturation is reached. Stir the “HVW super 3” on a magnetic stirrer while adding the saturated ammonium sulphate. At the end of the hour allow the material to equilibrate for 30 minutes.

Centrifuge the “HVW super 3” in the high speed centrifuge at 12,000×g for 20 minutes. Keep the pellet (LPS fraction). Resuspend the “LPS fraction” in cold 0.05M sodium phosphate buffer pH 7.2 at a ratio of 10 mL buffer for each 250 CFA plates that were used to produce the “vaccine washing” from which the “LPS fraction” was originally derived.

(Step L) Dialysis of “LPS fractions”. Dialyse the “LPS” fraction, using a 3,500 MW cut-off membrane, for 24-48 hours at 4° C. against 250-1,000 volumes of cold 0.05M sodium phosphate buffer pH 7.2. Change the buffer every 2-8 hours during dialysis. When complete, keep the “LPS dialysate” on ice until ready to assay the protein content.

Day 7 (Step M) Assaying protein content of “LPS dialvsate”. Use the Lowry protein assay to measure the protein content of each “LPS dialysate”. On the basis of the results dilute each “LPS dialysate” so that it contains 1 mg/mL of protein in 0.05M sodium phosphate buffer pH 7.2, and store in a sterile Schott bottle.

(Step N) Inactivation of vaccine. Add formaldehyde to each “LPS dialysate” so that the final concentration of formalin is 0.3%. Store the “formalinised LPS dialysate” at 4° C. for 3 days.

Day 10 (Step O) Sterility Checking Part 1. Carry out a basic sterility check on each “formalinised LPS dialysate” by inoculating 0.5 mL of each into 3 TSB tube broths. Place the “sterility check tubes” in the 37° C. incubator under aerobic conditions.

Day 14 (Step P) Sterility Checking Part 2 Check the “sterility check tubes” for absence of growth. If pure growth is absent proceed.

(Step Q) Storage of “formalinised Pilus/LPS dialvsate”. For longer term storage of the “formalinised LPS dialysate”, place it at minus 20° C.

Day 14 or Later (Step R) Adjuvantina Stage One Bring the “formalinised LPS dialysate” to 30° C. by placing it in a water bath. At the same time bring an equivalent volume of the adjuvant (Montanide ISA 206) to 30° C. in a water bath.

Pour the adjuvant into a large beaker which has been sterilised by autoclaving (S13). Use the Ika laboratory mixer with the 3-blade paddle to stir the adjuvant at 200 RPM. Add the “formalinised LPS dialysate” gradually over 2 minutes. Increase the speed to 2,000 RPM and maintain for 10 minutes. Store at 40° C. for 24 hours.

Day 15 or Later (Step S) Adiuvanting Stage Two. The day after Step L above, bring the “1 stage adjuvanted vaccine” to 30° C. in a water bath. Pour the warmed material into a large beaker which has been sterilised by autoclaving (S13). Use the Ika laboratory mixer with the 3-blade paddle to stir the material at 200 RPM for 2 minutes. Increase the speed to 2,000 RPM and maintain for 10 minutes. Store at 40° C. for 24 hours.

(Step T) Checking Quality of Emulsion. The day after Step M above, use a sterile Pasteur pipette to take a small aliquot of the “2nd stage adjuvante vaccine”. Fill a 250 mL beaker with approximately 200 mL of water. Place a drop of the aliquot onto the surface of the water. If the drop partially dilutes itself, giving a milky appearance to the water then it is water-in-oil-in-water and is acceptable. Store at 4° C. for 24 hours.

Day 16 or Later (Step U) Fitting. Working in the biological safety cabinet, use a funnel and measuring container which have been sterilised by autoclaving to measure out the appropriate volume of the vaccine. For 250 mL pillow packs this is 253-255 mL. Pour this material into pillow packs which have been sterilised by gamma radiation. Rubber stoppers and metal caps that have been sterilised by autoclaving are then used to close the top of each pillow pack. This process is then repeated until the appropriate number of pillow packs for the batch have been filled.

(Step V) Retention Sampling. Procure a retention sample.

(Step W) QC Sampling for Sterility Testing and Free Formalin Level Testing. Using the material left over at the end of the filling run, fill a 20 ml sample into a MacCartney bottle which has been sterilised by autoclaving. Use the same funnel and measuring container which were used to fill the rest of the run. Use this sample to perform sterility testing and free formalin level testing.

(Step X) Labelling. Label each batch. Store in refrigerator.

(Step Y) First Sterility Check. Four days after filling, carry out the first check on the “sterility test tubes” and “sterility test plates” as described in Testing for Sterility.

(Step Z) Second Sterility Check Seven days after filling, carry out the second check on the “sterility test tubes” and “sterility test plates” as described in Testing for Sterility.

(Step AA) Third Sterility Check. Eleven days after filling, carry out the third check on the “sterility test tubes” as described in Testing for Sterility.

(Step AB) Fourth Sterility Check. Fourteen days after filling, carry out the fourth check on the “sterility test tubes” as described in Testing for Sterility.

(Step AC) Product Use. The batch is acceptable for use if it satisfies the following specifications: Physical appearance: a milky creamy liquid in a plastic pillow pack labelled with the approved format label including the name “Anadis E. coli Vaccine”.

Sterility: absence of any indication of growth on plates or in tubes at the time of the sterility check and initial formal sterility test, or absence of any sign of growth during retesting as described in Testing For Sterility.

Potency: Greater than or equal to 1.0 mg of protein per mL of finished product.

Free Formalin Level: level in the QC sample is no greater than 0.002% w/v.

Emulsion quality: a drop of the emulsified vaccine placed on water partially dilutes itself, giving to the water a milky appearance.

### Part B

A composition for treatment or inhibition of Influenza A may be prepared by co-inoculation of a subject with 1 ml of antigen derived from Example 1 and 1 ml of LPS antigen derived from the procedure of Example 8. The two antigenic materials may be injected in separate injections 3 times into a pregnant dairy cow at 2 week intervals, with the last injection occurring 1 month before calving. The Influenza A inoculation may use 15 micrograms of the killed H1N1 influenza virus (PR8 mouse adapted strain) mixed with 1 ml of Montenide ISA adjuvant.

When co-vaccination is carried out, as above, the titre of blood samples may be five times greater than when vaccination is carried out with Influenza A antigens.

### Example 10

This example describes the production of high-titre antibodies in bovine colostrum that can inhibit hemagglutination and neutralize the infectivity of influenza virus.

Cattle were vaccinated with a range of experimental vaccines prepared using killed virus and adjuvants suitable for use in animals for food production.

Viruses of different hemagglutinin types are used to raise polyclonal antibody preparations (e.g. A/PR/8/34 (PR8) (H1N1), A/WSN RG/33 (WSN) (H1N1), A/Adachi/2/57 (Adachi) (H2N2), A/Singapore/1/57 (H2N2), A/duck/Hong Kong/836/80 (H3N1), A/Aichi/2/68 (Aichi) (H3N₂), A/Memphis/1/96 (Memphis) (H3N2), A/duck/Hokkaido/5/77 (H3N2), A/chicken/Hong Kong/37/78 (H3N2), A/duck/Hokkaido/8/80 (H3N8), A/Hong Kong/483/97 (H5N1), A/rgViet Nam/1194AHA/2004 (rgVNΔHA) (H5N1), A/swan/Hokkaido/67/96 (Hok67) (H5N3), A/swine/Hong Kong/10/98 (HK10) (H9N₂), A/duck/Hong Kong/W213/97 (W213) (H9N2), A/duck/Hokkaido/49/98 (H9N2), and A/gull/Maryland/704/77 (Maryland) (H13N6).

Polyclonal antibody preparations were successfully induced in the cattle and showed good hemagglutination inhibition (HI) and virus neutralization (VN) activity.

### Part A

Dilutions of polyclonal antibody preparations induced in cattle were tested in ELISA for binding to split influenza virus adsorbed to wells of polyvinyl plates using standard procedures.

Binding of specific anti-influenza antibodies was detected, and titres expressed as the reciprocal of the dilution giving an optical density of 0.2.

Table 1 demonstrates the production of high-titre antibodies in bovine colostrum that specifically bind H1N1 (PR8) influenza virus.

High-titre antibodies in bovine colostrum that specifically bind H3 hemagglutin- or H5 hemagglutin-influenza virus are also produced.

TABLE 1
Hemagglutination inhibition, ELISA antibody-binding titres and virus
neutralizing (VN) activity of anti-PR8 IgG and F(ab′)₂ preparations

| Purified | Ab HI | ELISA | titre VN |
|---|---|---|---|
| Sample | titre^{a} | (log₁₀)^{b} | titre^{c} |
| Anti-PR8 IgG | 1280 | 4.8 | 79,000 |
| Anti-PR8 F(ab′)₂ | 2560 | 4.0 | 400,000 |
| Non-immune IgG | <10 | 1.2 | <10 |
| Non-immune | <10 | 1.2 | <10 |
| F(ab′)₂ | | | |

^{a}HI titres are expressed as the reciprocal of the mean antibody dilution inhibiting 3 out of 4 HA units of virus.
^{b}Antibody titres are expressed as the reciprocal of the dilution giving an optical density of 0.2.
^{c}Virus neutralization activity is expressed as the reciprocal of the antibody dilution at which 50% of 50 plaque forming units were neutralized.

### Part B

Hemagglutination inhibition assays were performed with chicken red blood cells using standard procedures.

In brief, tests were performed in round-bottom 96-well microtiter plates at room temperature using 1% v/v chicken erythrocytes. Dilutions of purified antibody sample were prepared and 4 hemagglutinating (HA) units of virus were added. Following incubation, chicken erythrocytes were added and the ability of antibodies to inhibit virus-induced hemagglutination was assessed.

Polyclonal antibody preparations according to the present invention showed good HI activity (Table 1), indicating that the polyclonal antibodies include antibodies that specifically bind influenza hemagglutin and inhibit hemagglutination. In particular, antibody preparations were produced that specifically bound H1 hemagglutin and inhibited hemagglutination (e.g. see Table 1).

Antibody preparations are also produced that specifically bind H3 hemagglutin and inhibit hemagglutination, or bind H5 hemagglutin and inhibit hemagglutination.

### Part C

Virus neutralization activity was measured using standard procedures.

In brief, dilutions of polyclonal antibody preparation according to the present invention were mixed with a standard amount of virus and, following incubation for 45 mins, the mixtures were inoculated onto Madin-Darby canine kidney (MDCK) cell monolayers and overlaid with agarose. Three days later, plaque formation was assessed.

Polyclonal antibody preparations showed good viral neutralization activity (Table 1), indicating that polyclonal antibodies induced in cattle can bind and neutralize influenza virus.

### Example 11

This example describes high-titred antibodies from bovine colostrum prevent infection (to a level of 100% protection) with virulent influenza for at least 3 days following a single dose.

### Part A

Adult male mice (Balb-c) were treated intranasally with 1 mg of immune IgG or immune F(ab)′2 fragments according to the present invention or 1 mg of non-immune IgG or non-immune F(ab)′2 fragments

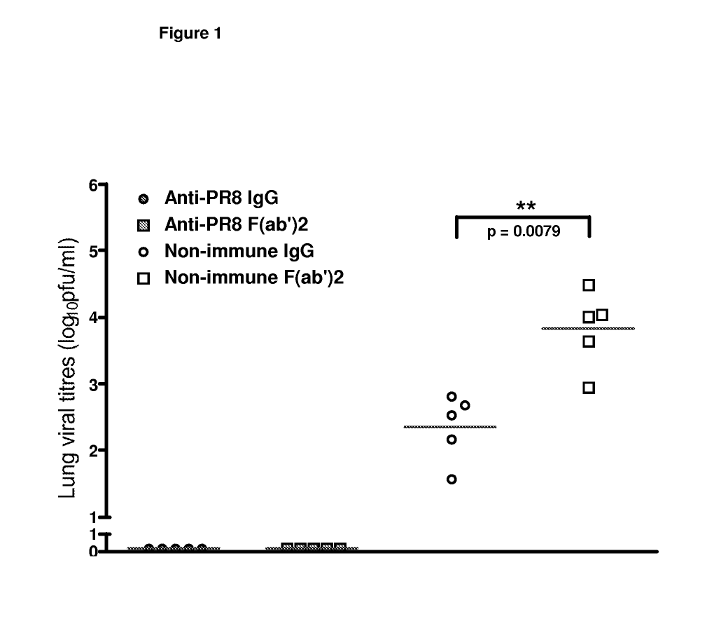

24 hours later, mice were infected with a lethal dose of H1NI (PR8) influenza confined to the upper respiratory tract, by a drop put on the nares. On day 1 post-infection, the mice were killed and their noses were collected and viral loads in the nasal turbinates were determined by plaque assay (FIG. 1).

Viral titres indicate that the high-titred antibodies from bovine colostrum prevent infection (to a level of 100% protection) with virulent influenza for 24 hours following a single dose.

### Part B

Adult male mice (Balb-c) were treated intranasally with 1 mg of immune IgG or immune F(ab)′2 fragments according to the present invention or 1 mg of non-immune IgG or non-immune F(ab)′2 fragments, or PBS alone.

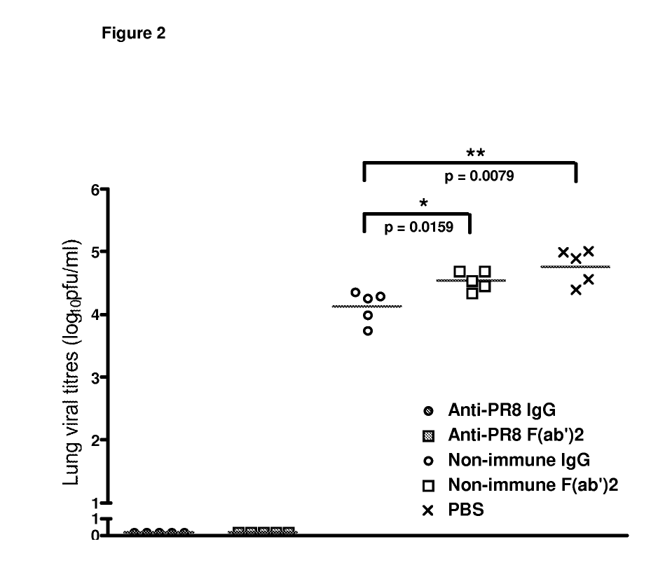

24 hours later, mice were infected with a lethal dose of H1NI (PR8) influenza confined to the upper respiratory tract, by a drop put on the nares. On day 5 post-infection, the mice were killed and their noses were collected and viral loads in the nasal turbinates were determined by plaque assay (FIG. 2).

100% of treated mice were protected by immune IgG and immune fragments.

Viral titres indicate that the high-titred antibodies from bovine colostrum prevent infection (to a level of 100% protection) with virulent influenza for 5 days post infection following a single dose.

There was a small effect of non-immune IgG but this effect was lost by day 5. This indicates that protection provided by antibodies from bovine colostrum was complete while other control treatments either did not prevent infection or allowed infection to rebound to significance.

### Part C

Adult male mice (Balb-c) were treated intranasally with 1 mg of immune IgG or immune F(ab)′2 fragments according to the present invention or 1 mg of non-immune IgG or non-immune F(ab)′2 fragments.

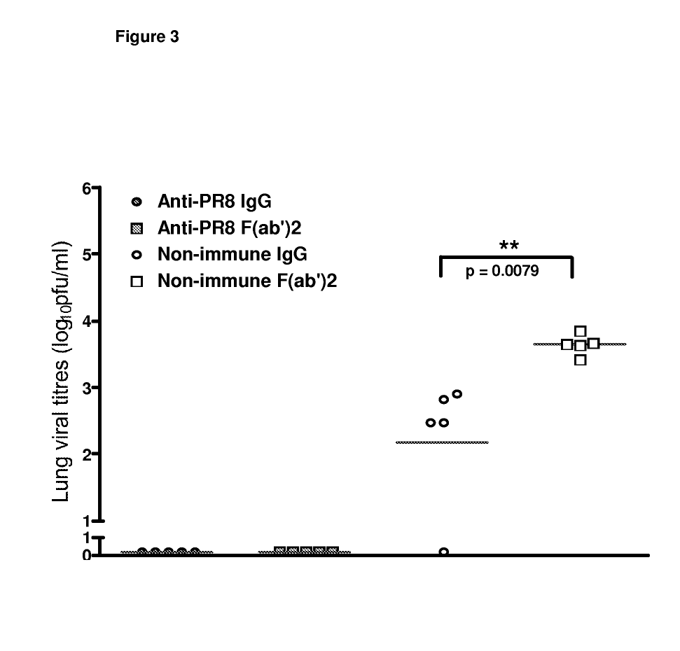

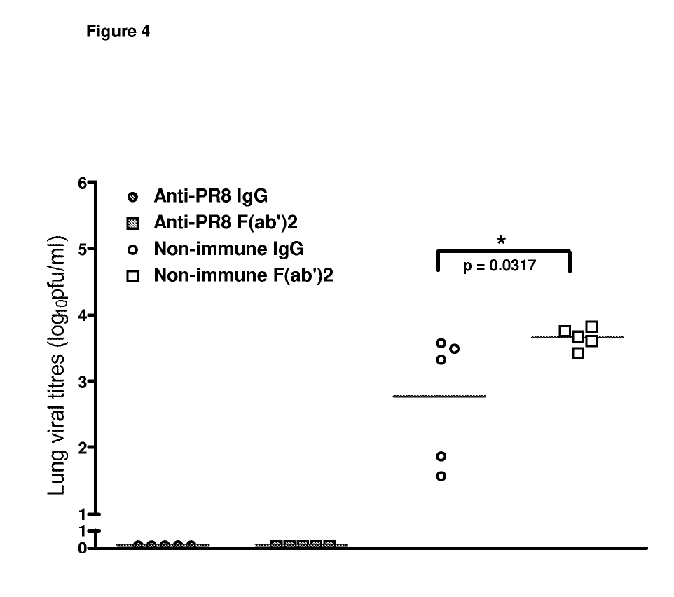

48, 72 and 168 hours later, mice were infected with a lethal dose of H1NI (PR8) influenza confined to the upper respiratory tract, by a drop put on the nares. On day 1 post-infection, the mice were killed and their noses were collected and viral loads in the nasal turbinates were determined by plaque assay (FIG. 3 and FIG. 4).

100% of treated mice were protected by immune IgG and immune fragments for two days (FIG. 3), and 100% of treated mice were protected by immune IgG and immune fragments for three days (FIG. 4).

80% of treated mice were protected by immune IgG and immune fragments for seven days (data not shown).

Viral titres indicate that the high-titred antibodies from bovine colostrum prevent infection (to a level of 100% protection) with virulent influenza for 3 days following a single dose.

### Example 12

The following example demonstrates the activity of IgG preparations in ameliorating upper respiratory tract infection with influenza virus.

adult male mice (Balb-c) were challenged with a lethal dose of H1NI (PR8) influenza confined to the upper respiratory tract. 24 hours following infection, the mice were treated intranasally with various doses (50 ug, 100 ug, 150 ug, and 200 ug) of immune IgG according to the present invention, or non-immune IgG.

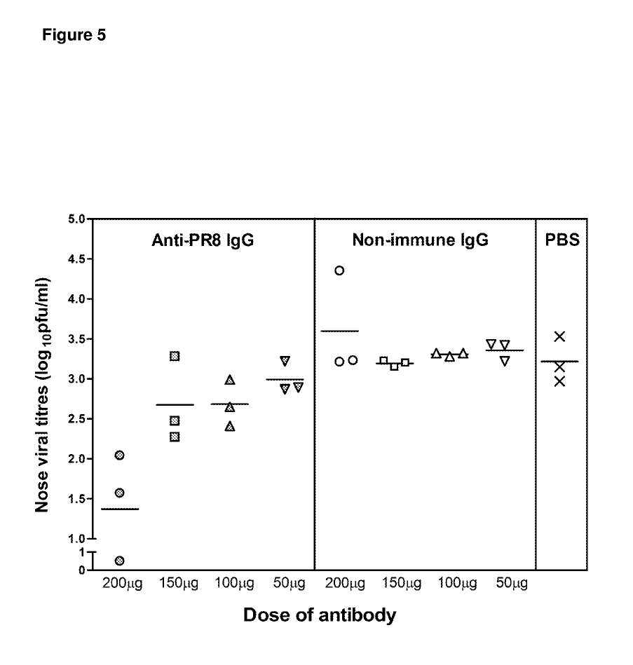

On day 5 post infection, viral loads in the nasal turbinates were determined by plaque assay (FIG. 5).

Significant reduction of viral loads were seen following treatment with specific immune IgG according to the present invention but not non-immune IgG. A significant effect was observed at 5 days post infection; 4 days after a single IgG treatment.

A reduction in nasal shedding is expected to lessen symptom severity in individuals and also decrease virus transmission within populations.

### Example 13

The following example demonstrates the activity of IgG preparations in ameliorating pulmonary infection with influenza virus.

33 adult male mice (Balb-c) were challenged with a sub-lethal dose of H1NI (PR8) influenza as a total respiratory tract infection and 24 hrs later the mice were treated intranasally with various doses (100 ug, 200 ug, 500 ug, 800 ug and 1000 ug) of immune IgG according to the present invention or non-immune IgG.

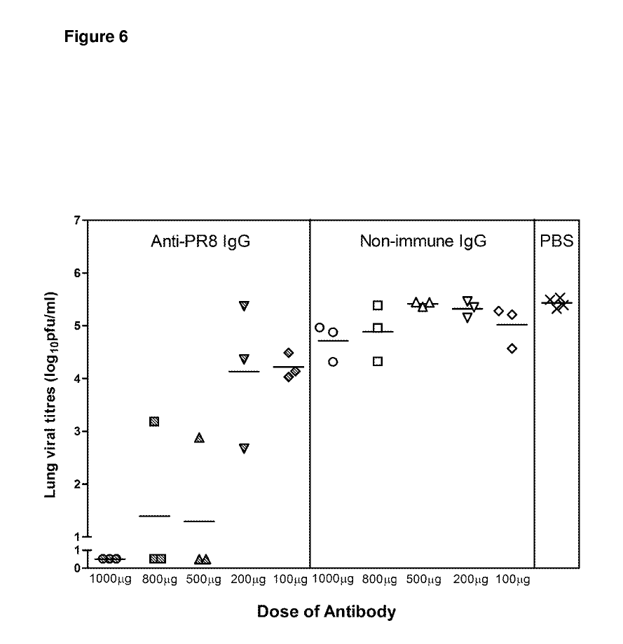

On day 5 post infection, viral loads in the lungs were determined by plaque assay (FIG. 6).

Mice were infected with a sub-lethal dose of PR8 as a total respiratory tract infection and 24 hrs later the mice were treated intranasally with various doses of immune or non-immune IgG.

Significant reduction of pulmonary viral loads were seen following treatment with specific immune IgG according to the present invention but not non-immune IgG. A significant effect was observed at 5 days post infection; 4 days after a single IgG treatment. At high concentrations of immune IgG, virus was undetectable in the lungs of individual animals within the group.

This demonstrates in many animals, treatment with antibodies of the present invention halted an existing infection, rather than delaying clinical development.

### Example 14

The following example demonstrates the activity of IgG preparations protection against lethal challenge and clinical signs of infection.

Mice were challenged with a lethal dose of H1NI (PR8) influenza as a total respiratory tract infection and 24 hrs later the mice were treated intranasally with 1 mg of immune IgG according to the present invention or non-immune IgG, or PBS.

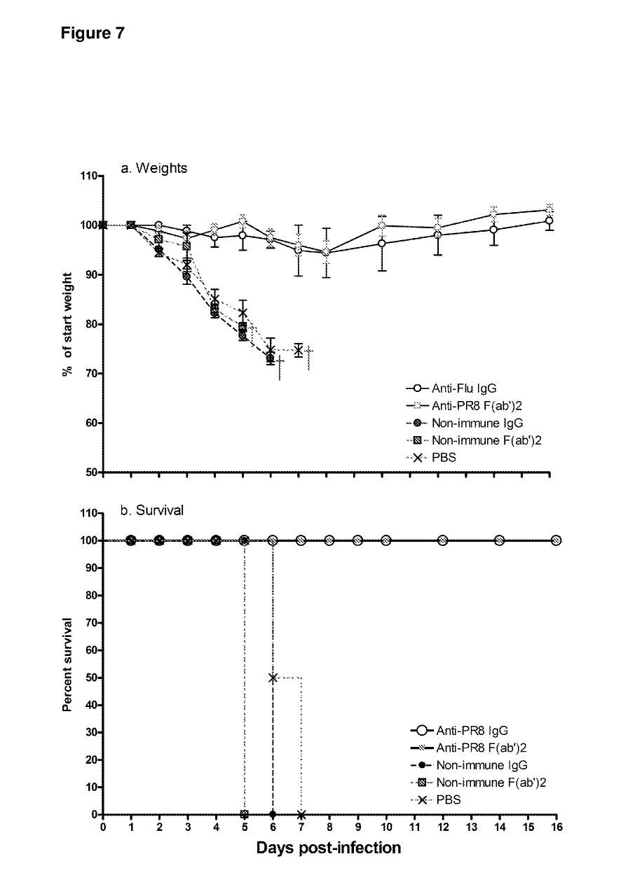

Mice were monitored daily for clinical signs and their weights determined over a 16-day period (FIG. 7a). Mice were culled at the humane endpoint and a survival curve plotted (FIG. 7b).

The course of lethal disease was stopped in mice treated with the immune IgG preparations, while the disease proceeded in all groups treated with non-immune preparations. There was no significant weight loss in groups treated with immune IgG.

Both immune IgG and F(ab′)₂ preparations according to the present invention not only protected mice from death, but also prevented clinical signs of infection (not shown) including weight loss after a single administration.

1. A method for treatment or inhibition of influenza infection in one or more subjects comprising applying to a surface selected from the group consisting of air filters, sick room surfaces and respiratory mucosal membranes at least one immune material selected from the group consisting of antibodies and fragments thereof which bind a least one Influenza A antigen selected from the group consisting of H1, H3 and H5, said immune material being derived from hyperimmune milk products, said hyperimmune milk products being prepared by inoculation of mammals with antigen comprising at least one Influenza A antigen selected from the group consisting of H1, H3 and H5.

2. A method of treatment or inhibition of influenza infection in a human subject comprising administering by inhalation a composition comprising immune material selected from the group consisting of antibodies and fragments thereof which bind a least one Influenza A antigen selected from the group consisting of H1, H3 and H5, said immune material being derived from hyperimmune milk products prepared by inoculation of mammals with antigen comprising a least one Influenza A antigen selected from the group consisting of H1, H3 and H5.

3. A method according to claim 2, wherein said immune material is derived from hyperimmune colostrum.

4. A method according to claim 2, wherein the hyperimmune milk product is prepared by further inoculating the mammals with lipopolysaccharide (LPS).

5. A method according to claim 2, wherein the immune material is prepared by inoculating mammals with an influenza A virus antigen selected from the group consisting of H1, H3 and H5 in the form of an attenuated virion, virus like particle, viral protein or epitopes from portions or assemblies of viral proteins.

6. A method according to claim 2, wherein the antigen comprising at least one said influenza A antigen is a split antigen.

7. A method according to claim 2, wherein the immune material is adsorbed on a particulate carrier of a size in the range of from 50 to 120 microns.

8. A method according to claim 2, wherein the immune material is administered as an aerosol.

9. A method according to claim 2, wherein the total of IgA, IgE and IgM is less than 20% of total Immunoglobulin and the IgG component constitutes at least 50% of IgA, IgE, IgG and IgM.

10. A method according to claim 1, wherein said immune material derived from hyperimmune milk products is hyperimmune colostrum in dried form.

11. A method according to claim 1, wherein the surface is a filter on which the immune material is adsorbed to inhibit infection of people with the influenza A virus which may be present in the airstream.

12. A method according to claim 1, wherein the immune material recognises at least two of said H1, H3 and H5 antigens.

13. A method according to claim 3, wherein the method is for treatment of influenza infection and the subject is suffering from influenza A infection.

14. A method according to claim 2, wherein the immune material is in the form of a unit dose for inhalation comprising at least 1 mg of antibody.

15. A composition for the treatment or inhibition of influenza infection by inhalation comprising at least one immune material selected from the group consisting of antibodies derived from milk products and fragments thereof which bind a least one Influenza A antigen selected from the group consisting of H1, H3 and H5.

16. A composition for the treatment or inhibition of influenza infection according to claim 15, wherein the immune material is absorbed onto a solid carrier for administration by dry powder inhalation.

17. A composition for the treatment or inhibition of influenza infection according to claim 15, wherein the immune material is adsorbed on a particulate carrier of a size in the range of from 50 to 120 microns.

18. A composition for the treatment or inhibition of influenza infection according to claim 15, wherein the immune material recognises at least two of said H1, H3 and H5 antigens.

19. A composition for the treatment or inhibition of influenza infection according to claim 15, wherein the immune material is in the form of a unit dose for inhalation comprising at least 1 mg of antibody.

20. A composition for the treatment or inhibition of influenza infection according to claim 15, wherein the composition is absorbed onto an air filter material selected from the group consisting of personal air filters, air conditioning filters and building ventilation filters.
